(19) United States
(12) Patent Application Publication
(10) Pub. No.: US 2010/0030070 A1
(43) Pub. Date: Feb. 4, 2010

(54) SYSTEM AND METHOD FOR INJECTING CONTRAST PRODUCTS

(76) Inventors: Hervé Duffour, Lausanne (CH); Frédéric Neftel, Lausanne (CH)

Correspondence Address: NIXON & VANDERHYE, PC, 901 NORTH GLEBE ROAD, 11TH FLOOR, ARLINGTON, VA, 22203, US

(21) Appl. No.: 12/304,207

(22) PCT Filed: Jun. 13, 2007
(86) PCT No.: PCT/IB2007/052236

§ 371 Date: Oct. 14, 2009

(30) Foreign Application Priority Data
Jun. 16, 2006 (EP) 06115584.2

(51) Int. Cl. A61B 6/00

(52) U.S. Cl. 600/431

(57) ABSTRACT

The invention concerns a system for injecting contrast product comprising an injecting mechanism, at least two reservoirs operatively associated with the injecting mechanism, said reservoirs being designed to contain at least one contrast product and one rinsing product respectively; and a control device comprising programming means, which is operatively associated with the injecting mechanism and capable of operating for programming selectively one or more injection process phases. The system is characterized in that it comprises a triggering member operatively associated with the control device so that the control device is capable of programming at least one injecting phase of the contrast product at a predetermined flow rate followed by a rinsing phase at another predetermined flow rate and a programmed duration for injecting the contrast product based on a signal given during the injection by said triggering member when the contrast product is visualized in a region of interest. The invention also concerns a method for using said system as well as various applications thereof.

### FIELD OF THE INVENTION

The invention relates to the injection of contrast products. It relates more specifically to the injection of contrast products followed by an injection of a rinsing product.

### STATE OF THE ART

Various systems and methods are known for injecting contrast products. See in particular the American patent U.S. Pat. No. 6,385,483 B1 and the American patent application US 2003/0216643 A1.

### General Explanation of the Invention

The problem that the present invention proposes to address lies in best use of contrast products for requirements associated with examination, in particular by reducing the necessary quantity of contrast product (and at the same time the side effects associated with the contrast products) while synchronizing the injection with the acquisition sequence of the examination proper. Thus, the invention makes it possible to guarantee a better success for the examination procedures by guaranteeing that the contrast product is indeed present throughout the necessary time during the examination sequence, while minimizing the quantity injected.

In the invention, the solution to the abovementioned problem consists of a system for injecting contrast product comprising an injection mechanism, at least two reservoirs operatively associated with the injection mechanism, said reservoirs being designed to contain respectively at least one contrast product and one rinsing product; and a control device, comprising programming means, which is operatively associated with the injection mechanism and capable of operating to selectively program one or more injection procedure phases; the system being characterized in that it comprises a triggering member operatively associated with the control device so that the control device is designed to program at least one contrast product injection phase at a determined flow rate followed by a rinsing phase at another determined flow rate and a non-determined time for the injection of the contrast product which is dependent on a signal given during the injection by said triggering member when the contrast product is viewed in a region of interest.

According to one embodiment of the invention, the system comprises means to enable a user to program the image acquisition time and/or the time for which the contrast product must be present in the image acquisition area.

Alternatively, the acquisition time can be determined automatically, on the basis of certain basic data supplied to the system.

It should be noted that, in general, the image acquisition area does not correspond to the area of interest, the latter being situated upstream of the image acquisition area.

The terms “upstream” and respectively “downstream” should be understood to mean the region that comes before, and respectively after, the region concerned which is passed through by the liquid (contrast or rinsing product) in the patient's vascular system.

It should also be noted that the image acquisition area can be static or dynamic. More often than not, it is a dynamic area corresponding to an area scanned by the imaging appliance during the image acquisition sequence. In the case of a dynamic acquisition area, the area where the contrast product is present is also dynamic since it must follow the acquisition area.

According to another embodiment of the invention, the system comprises processing means which, during the image acquisition time, incorporate any time needed to reposition the imaging appliance before the start of image acquisitions.

The triggering member can be activated manually and/or automatically.

If the activation is automatic, it can be by an imaging device such as the central computer of the CT scanner.

Advantageously, the system comprises security means which are designed to limit a maximum injection volume of contrast product during a phase. This maximum volume can be programmed for each patient or calculated automatically according to other programmed parameters. This volume is notably useful in the case where the signal is triggered manually erroneously (too late or not at all).

The invention also relates to a method that uses the system as defined previously. The method is characterized in that it comprises at least one phase of contrast product injection at a determined flow rate followed by a rinsing phase at another determined flow rate, the injection time of the contrast product being dependent on the signal given during the injection by the triggering member when the contrast product is viewed in a region of interest.

Preferably, the activation of the triggering member also initiates an image acquisition process.

According to one embodiment of the invention, the injection time of the contrast product is greater than the time needed for the viewing in the observation area, said injection time being calculated according to the image acquisition time.

According to a variant, account is taken of the volume of contrast product situated in the vascular system of the patient (between the injection point and the area of interest) by analysis of the time needed for the contrast product to appear in the area of interest from the start of the injection, in order to minimize the volume of contrast product injected into the patient by the use of at least a part of this volume of buffer contrast product located in the vascular system which will be pushed by the rinsing product, the injection sequence for which will be advanced, before the end of the acquisition sequence, while making it possible to ensure that the contrast product is present in the area that has to be examined throughout the duration of the acquisition sequence.

According to another embodiment of the invention, the flow rate of the rinsing product is identical to that of the contrast product at least throughout the remaining time of the acquisition sequence, the injection of the rinsing product being advanced by a time that is at most equal to that defined between the start of the injection of contrast product and the instant at which the signal has been triggered.

Advantageously, the flow rate of the rinsing product is reduced after the end of the image acquisition sequence.

According to a variant, the flow rate of the rinsing product is reduced gradually.

According to another embodiment of the invention, the total volume of rinsing product injected is programmed initially.

According to another embodiment of the invention, the total volume of rinsing product injected is calculated according to preprogrammed parameters.

The invention finally relates to various uses of the system and of the method as defined above.

According to one embodiment, the triggering member is activated at the moment when the contrast product enters into the area of interest.

This activation can be performed by a user, for example a technician, who himself views on a screen the arrival of the contrast product in the area of interest.

Alternatively, the triggering member is activated automatically, following a signal transmitted by the imaging device when the contrast product is detected in the area of interest.

One particularly interesting field of use is imaging by CT scanner.

The invention can notably be advantageously used in angiographic CT (ACT) imaging procedures. According to such an embodiment of the invention, the acquisition sequence corresponds to that of the images of the heart. In such a case, the signal is given when the contrast product appears in a region situated at the output of the left ventricle. According to such an embodiment of the invention, the rinsing sequence is determined so as to guarantee that the concentration of contrast product present in the right heart during the acquisition phase is less than a determined threshold. Preferably, the quantity of contrast product in the right heart is minimized.

Another field of use of the invention is lung imaging. According to such an embodiment of the invention, the area of interest is preferably situated in an area at the output of the right ventricle. Advantageously, the progression of the appearance of the rinsing product in the lung is calculated precisely according to the acquisition sequence.

The present invention presents several advantages, in particular of reducing the quantity of contrast product to be used for the acquisition of an image, so lowering the toxicity and reducing the costs associated with the production of an image, while guaranteeing the presence of contrast product throughout the necessary phase of the acquisition of the image and while reducing the toxic effects associated with the total quantity of contrast product injected into the patient.

### DETAILED EXPLANATION OF THE INVENTION

The invention will be better understood hereinbelow from detailed examples.

In the case of an imaging by ACT scanner, it is important to view coronaries of the heart. For this, it is essential to ensure that the contrast product is indeed present in the coronaries throughout the time when the images are acquired (typically between 6 and 12 seconds for a 64-slice CT scanner) . At the same time, it is preferable to ensure that the right part of the heart contains no or little contrast product, in order to be able to view the septum and calculate the cardiac ejection fractions.

Thus, it is routine today to program volumes of contrast product for an adult of the order of 70 to 80 ml at a flow rate of 3 to 5 ml/s, followed by a rinsing sequence at one and the same flow rate for a volume of the order of 30 to 50 ml. These data make it possible to guarantee the success of the examination regardless of the heart rate, the circulatory volume and the acquisition time from the presence of a signal at the output of the left ventricle (typically at the level of the aortic arch), without optimizing the quantity of contrast product injected, that is, with a risk for the patient associated with the side effects that are directly proportional to the volume of contrast product injected.

In the context of the invention, the dose of contrast product is preferably limited (typically changing from 70-80 ml to 40-60 ml) by not programming the injected volume but rather the time during which the contrast product must be present in the left heart (generally the time during which the image acquisition is done). This time must, if necessary, take account of the repositioning time of the scanner (approximately 1 to 3 seconds) from the moment when the signal is documented at the output of the left heart. Thus, the injection of contrast product will be done throughout the time needed to perform a correct examination, so avoiding insufficient injection sequences (for which the examination risks having to be repeated) and excessive quantities of injected contrast product. Furthermore, it is important to ensure a draining of contrast product from the right heart during this acquisition phase, which is obtained by the injection of rinsing product sufficiently rapidly after the appearance of contrast product in the area of interest (generally, the repositioning time of the scanner is greater than the time needed for the rinsing product to arrive in the right heart, so making it possible to initiate the rinsing sequence according to the arrival of contrast product at the output of the left heart while taking into account the time that would have been needed for the contrast product to arrive at the left heart from the injection point to ensure that contrast product is present in the left heart throughout the acquisition sequence). Ideally, the injection sequence of the rinsing product must be calculated to minimize the total quantity of contrast product injected into the patient, while ensuring that contrast product is still present in the left heart throughout the image acquisition time and that a minimum of contrast product is present in the right heart throughout this same image acquisition period.

By proceeding in this way, it is possible to initiate the rinsing sequence at the right moment, while minimizing the injection of contrast product for which the injection time does not generally overrun the end of the image acquisition sequence. Furthermore, the contrast product injection flow rate, throughout the injection, will preferably remain fixed, whereas the injection flow rate of the rinsing product, after the end of the contrast product injection sequence, may, if necessary, be reduced. Preferably, however, the flow rate of the rinsing product remains fixed at least throughout the image acquisition time. These injection and contrast product flow rates can, however, if necessary, vary, during each of the phases. The contrast and rinsing product injection sequences are, preferably, always sequential, no dilution between the contrast product and the rinsing solution in principle being necessary.

1. A system for injecting contrast products comprising an imaging device suitable for viewing a contrast product in an area of interest, an injection mechanism, at least two reservoirs operatively associated with said injection mechanism, said reservoirs being designed to contain respectively at least one contrast product and one rinsing product; a control device, comprising programming means, which is operatively associated with the injection mechanism and capable of operating to selectively program one or more injection procedure phases; the system being characterized in that it comprises a triggering member operatively associated with the control device and with the imaging device, said triggering member being designed to be activated when the contrast product is viewed in a region of interest.

2. The system as claimed in claim 1, comprising means for programming an image acquisition time from the activation of the triggering member.

3. The system as claimed in claim 2, comprising processing means which, during the image acquisition time, incorporate any time needed to reposition the imaging appliance before the start of image acquisitions to calculate the injection time.

4. The system as claimed in claim 1, in which the triggering member is designed to be activated manually.

5. The system as claimed in claim 1, in which the triggering member is designed to be activated automatically.

6. The system as claimed in claim 5, characterized in that it can be activated directly by the imaging device.

7. The system as claimed in claim 1, comprising security means which are designed to limit a maximum injection volume of contrast product during a phase, said maximum volume preferably being defined according to the patient.

8. A method using a system as defined in claim 1, for the injection of contrast products and the acquisition of images, said method being characterized in that it comprises at least one phase of contrast product injection at a determined flow rate, without either the volume or the time being determined in advance, followed by a rinsing phase at another determined flow rate, the injection time of the contrast product being dependent on a signal given during the injection by the triggering member when the contrast product is viewed in a region of interest, in order to make it possible to acquire contrasted images in an area called “image acquisition area”.

9. The method as claimed in claim 1, characterized in that the initiation of the triggering member also initiates an image acquisition process, said initiation being performed manually or automatically.

10. The method as claimed in claim 8, in which the injection time of the contrast product from the triggering signal is dependent on the time needed for the presence of contrast product in said image acquisition area, said injection time being calculated according to the image acquisition time.

11. The method as claimed in claim 10, in which account is taken of the volume of buffer contrast product situated in the vascular system of the patient (between the injection point and the area of interest), this volume being deduced by analysis of the time needed for the contrast product to appear in the area of interest from the start of the injection, in order to minimize the volume of contrast product injected into the patient because of the use of at least a part of this volume of buffer contrast product which will be pushed by the rinsing product before the end of the acquisition sequence in such a way that contrast product is present in the area that has to be examined throughout the duration of the acquisition sequence.

12. The method as claimed in claim 11, in which the rinsing product is injected at the earliest Tr seconds before the end of the acquisition sequence, Tr representing the time between the start of the injection of contrast product and the signal, in order to ensure that there is no rinsing product in the area situated downstream of the area of interest throughout the acquisition.

13. The method as claimed in claim 11, in which the rinsing product is injected early enough after the triggering of the signal in order to ensure that a certain territory situated upstream of the area of interest contains a low level of contrast product, but late enough to ensure that the part of the acquisition area which is situated downstream of the image acquisition area contains a sufficiently high level of contrast product throughout the acquisition.

14. The method as claimed in any claim 10, in which the flow rate of the rinsing product is identical to that of the contrast product at least throughout the remaining time of the acquisition sequence, the injection of the rinsing product being advanced by a time that is at most equal to that defined between the start of the injection of contrast product and the instant at which the signal has been triggered.

15. The method as claimed in claim 8, in which the flow rate of the rinsing product is reduced after the end of the image acquisition sequence.

16. The method as claimed in claim 15, in which the reduction is gradual.

17. The method as claimed in claim 8, in which the total volume of rinsing product injected is programmed initially.

18. The method as claimed claim 8, in which the total volume of rinsing product injected is calculated according to preprogrammed parameters.

19. The use of a system as claimed in claim 1, characterized in that the triggering member is activated manually or automatically at the moment when the contrast product is viewed in the area of interest.

20. The use of a system as claimed in claim 1 for imaging by CT scanner.

21. The use of a system as claimed claim 1 for angiography CT imaging procedures.

22. The use as claimed in claim 21, in which the acquisition sequence corresponds to that of the images of the heart.

23. The use as claimed in claim 22, in which the signal is given when the contrast product appears in a region situated at the output of the left ventricle.

24. The use as claimed in claim 20, in which the rinsing sequence is determined so as to guarantee a certain concentration of contrast product present in the right heart during the acquisition phase which is less than a determined threshold.

25. The use as claimed in claim 24, in which the quantity of contrast product in the right heart is minimized.

26. The use of a system as claimed in claim 1 for imaging the lung.

27. The use as claimed in claim 26, in which the area of interest is situated in an area at the output of the right ventricle.

28. The use as claimed in claim 26, in which the progression of the appearance of the rinsing product in the lung is calculated precisely according to the acquisition sequence.
